(19) United States
(12) Patent Application Publication
(10) Pub. No.: US 2024/0316310 A1
(43) Pub. Date: Sep. 26, 2024

(54) NEUROPERCEPTUAL FEEDBACK IN EXTENDED REALITY ENVIRONMENTS APPARATUSES, SYSTEMS, AND METHODS

(71) Applicants: University of Washington, Seattle, WA (US); SEATTLE CHILDREN'S HOSPITAL DBA SEATTLE CHILDREN'S RESEARCH INSTITUTE, Seattle, WA (US)

(72) Inventors: Courtnie Paschall, Seattle, WA (US); Jeffrey Andrew Herron, Seattle, WA (US); Emmanuel Tanumihardja, Seattle, WA (US); Rajesh P.N. Rao, Seattle, WA (US); Jason Scott Hauptman, Seattle, WA (US); Jeffrey G. Ojemann, Seattle, WA (US)

(21) Appl. No.: 18/608,428

(22) Filed: Mar. 18, 2024

Related U.S. Application Data
(60) Provisional application No. 63/491,236, filed on Mar. 20, 2023.
(60) Provisional application No. 63/623,128, filed on Jan. 19, 2024.

(51) Int. Cl. A61M 21/02

(52) U.S. Cl. CPC A61M 21/02; A61M 2205/04; A61M 2205/33; A61M 2210/0693; A61M 2230/63

(57) ABSTRACT

An extended reality (XR) neural perceptual feedback (NPF) system includes an XR system and a neural interface. The XR system presents a virtual environment to the user and monitors the user's interactions with the virtual environment. Based on the interaction with the virtual environment, the neural interface applies stimulation to the nervous system of the user. For example, a stimulation pattern may be selected based on the type of interaction, what type of virtual object the interaction was with, or combinations thereof. The stimulation may cause a percept in the user, allowing them to have perceptual feedback from interacting with the virtual environment. The neural interface may also be used to direct interactions within the virtual environment, for example using neural signal decoding to determine how to update the virtual environment.

### CROSS-REFERENCE TO RELATED APPLICATIONS

This application claims the benefit of benefit under 35 U.S.C. § 119 of the earlier filing date of U.S. Provisional Application Ser. No. 63/491,236 filed Mar. 20, 2023 and U.S. Provisional Application Ser. No. 63/623,128 filed Jan. 19, 2024, the entire contents of which are hereby incorporated by reference in their entirety for any purpose.

### STATEMENT OF FEDERALLY SPONSORED RESEARCH AND DEVELOPMENT

This invention was made with government support under EEC-1028725 awarded by the National Science Foundation. The government has certain rights in the invention.

### TECHNICAL FIELD

Embodiments of the disclosure relate generally to nervous system stimulation, and particularly, to neuroperceptual feedback.

### BACKGROUND

There has been growing interest in neural interfaces, such as brain computer interfaces (BCI). Some neural interfaces are capable of applying a stimulation to the nervous system of the user. For example, in direct electrical stimulation (DES) of the human cortex, electrical current is applied to the cortex by implanted, intracranial electrodes, such as electrocorticography (ECOG) or stereo-electroencephalography (sEEG). ECOG electrodes are implanted subdurally as flat grids or strips to record from the surface of the brain, while sEEG electrodes are inserted as vertical probes through the cortex to record from deeper brain structures. DES from either of these intracranial arrays may evoke perceivable sensations, called percepts, which are characterized by the function of the neural tissue being stimulated. For example, suprathreshold stimulation of the hand region of somatosensory cortex (S1) is known to elicit a percept localized on the contralateral hand. Although evoked percepts may be reliably discerned and localized, their subjective descriptions tend to underscore the artificial, strange, and unfamiliar nature of the percepts themselves.

Extended reality (XR) systems, such as virtual reality (VR) or augmented reality (AR) systems, are useful for presenting a user with an immersive virtual environment. Users may find it easier to conceptualize and interact with the virtual environment in a manner analogous to a real environment thanks to the immersive nature of the XR system. Feedback, such as haptic feedback (e.g., from vibration units in hand held controllers) may also be useful to help increase immersion. There may be a variety of applications where it may be useful to apply feedback via neural stimulation based on interaction with the virtual environment.

### SUMMARY OF THE INVENTION

In at least one aspect, the present disclosure relates to a method. The method includes presenting a virtual environment to a user with an extended reality (XR) system, determining an interaction of the user's with the virtual environment, generating a stimulation pattern responsive to the user's interaction with the virtual environment, and applying stimulation to the user's nervous system with a stimulation system based on the stimulation pattern.

The method may include applying a perceptual stimulation to the user's nervous system with the stimulation system. The method may include applying the stimulation to a sensory region of the user's brain. The method may include applying the stimulation through implanted electrodes. The method may include selecting the stimulation pattern based on a type of the interaction, a type of virtual object being interacted with, or combinations thereof.

The method may include generating the stimulation pattern responsive to the user grasping a virtual object in the virtual environment. The method may include generating a first stimulation pattern responsive to grasping a first type of virtual object and generating a second stimulation pattern responsive to grasping a second type of object. The method may include not generating the stimulation pattern or applying the stimulation responsive to grasping a third type of virtual object. The method may include training the user's nervous system to associate the stimulation with the interaction performed by the user in the virtual environment. Applying the stimulation may include modulating an amplitude of a sequence of stimulation pulses over time based on the stimulation pattern. The method may include receiving information from sensors which measure activity in the user's nervous system, decoding the information, and determining the interaction with the virtual environment based on the decoded information.

In at least one aspect, the present disclosure relates to a system. The system includes an extended reality (XR) system which presents a virtual environment to a user, a neural interface which delivers a stimulation to a nervous system of the user based on a stimulation pattern, and a computing system which generates the stimulation pattern responsive to an interaction of the user with the virtual environment and provides the stimulation pattern to the neural interface. The neural interface applies the stimulation pattern through the at least one electrode to the nervous system of the user.

The XR system may include at least one controller configured to allow the user to interact with the virtual environment. The system may include at least one sensor which measures activity in the nervous system of the user. The computing system may include a decoder which determines an interaction with the virtual environment based on decoding the measured activity from the sensor. The XR system may include a headset which is worn by the user. The at least one electrode may be implanted in the user.

In at least one aspect, the present disclosure relates to a system which includes an extended reality (XR) headset, a neural interface including at least one stimulation electrode, at least one processor, and computer readable non-transitory media loaded with instructions. The instructions, when executed by the at least one processor cause the system to generate a virtual environment and provide information related to the virtual environment to the XR system, receive input information from the XR system, determine an interaction with the virtual environment based on the input information, and provide a stimulation pattern to the neural interface responsive to the interaction. The neural interface applies the voltage to the at least one stimulation electrode based on the stimulation pattern.

The system may also include at least one controller which generates at least part of the input information. The input information may include motion tracking information. The system may also include at least one sensor which measures activity in a user's nervous system. The instructions, when executed by the at least one processor may further cause the system to decode the measured activity, where the decoded activity is at least part of the input information.

The at least one stimulation electrode may be implanted in a user. The at least one stimulation electrode may be implanted in a sensory region of the user's brain. The instructions, when executed by the at least one processor, may further cause the system to select the stimulation pattern from a plurality of stimulation patterns, where the selection is based on a type of the interaction, a type of virtual object the interaction is with, or combinations thereof.

### BRIEF DESCRIPTION OF THE DRAWINGS

FIG. 1 is a representation of an example extended reality neuroperceptual feedback (XR-NPF) system according to some embodiments of the present disclosure.

FIG. 2 shows graphs of example stimulation patterns according to some embodiments of the present disclosure.

FIG. 3 shows a pair of images which represent views of an example application for binary discrimination according to some embodiments of the present disclosure.

FIG. 4 shows a set of images which represent views of an example application for ternary discrimination according to some embodiments of the present disclosure.

FIG. 5 is a flow chart of a method according to some embodiments of the present disclosure.

FIG. 6 is a diagram of example electrode localizations for each subject according to some embodiments of the present disclosure.

FIG. 7 is an example view of a decoder according to some embodiments of the present disclosure.

### DETAILED DESCRIPTION

The following description of certain embodiments is merely exemplary in nature and is in no way intended to limit the scope of the disclosure or its applications or uses. In the following detailed description of embodiments of the present apparatuses, systems and/or methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The following detailed description is therefore not to be taken in a limiting sense, and the scope of the disclosure is defined only by the appended claims.

An extended reality (XR) system uses one or more multimedia systems to present a virtual environment to a user. For example, an XR system might include one or more displays to present an image of the virtual environment, one or more speakers to present sounds of the virtual environment and so forth. The user in turn, may interact with the virtual environment in one or more ways. For example, a controller, motion tracking, voice commands, other techniques, or combinations thereof may be used to interact with the virtual environment, and the presented virtual environment may be updated accordingly. An example XR system may include a headset with two displays that present a stereoscopic image of a virtual environment as if the user was present in the environment (e.g., from a first person view). The user may operate a handheld controller (e.g., pushing a thumbstick) to ‘move’ their point of view through the environment, causing the XR system to update the image displayed to the user. Another example interaction may involve moving the user's hand (or using a controller) to move a virtual representation of the user's hand (or an avatar controlled by the user in the virtual world) in order to manipulate objects in the virtual environment (e.g., pick up or move an object).

However, XR systems may be limited in the manner in which they can present information to the user. For example, XR systems may generally be audiovisual in nature. Recent advances have been made in neural interfaces, such as brain computer interfaces (BCI), which apply stimulation to a user's nervous system. For example, implanted electrodes in a user's brain may apply a sequence of electrical signals to induce activity in the user's nervous system. Some stimulation patterns, stimulated regions, or combinations thereof, may lead to percepts, or sensations which are perceivable by the user. These percepts may be mappable onto existing sensations (e.g., taste, touch, smell, vision, sound, etc.) or may take other forms. It may be useful to combine the virtual environments generated by an extended reality system with the ability to use a neural system to cause percepts in the user.

The present disclosure relates to apparatuses, systems, and/or methods for extended-reality neural-perceptual feedback. An XR-NPF system includes an XR system and a neural interface system. The XR system generates a virtual environment and presents it to the user and determines interactions that the user performs with the virtual environment. Based on those interactions, the XR-NPF system generates one or more stimulation patterns. The neural interface applies those stimulation patterns to the user's neural system (e.g., via direct electrical stimulation of the user's brain via implanted electrodes). The stimulation pattern causes a percept in the user which may act as neuroperceptual feedback (NPF) to the user. In an example application, a stimulation pattern may be generated and applied when the user causes their avatar to ‘touch’ an object in the virtual environment This stimulation may give the user perceptible feedback for their interaction with the virtual environment. In some embodiments, the stimulation may give the user the sensation of touch (and/or the user may be trained to associate the percept with the sense of touch). In some embodiments, different stimulation patterns, and thus different percepts, may be generated based on a type of interaction, a type of object being interacted with, or combinations thereof. For example interaction with a first type of object (e.g., a ‘soft’ virtual object) may cause a first stimulation pattern to be generated and applied (and a first percept to be experienced) while interaction with a second type of object (e.g., a ‘sharp’ virtual object) may cause a second stimulation pattern to be generated and applied (and a second percept to be experienced). The user may become able to distinguish the types of virtual object based on the stimulation induced sense of ‘touch’ alone, even without other indications (e.g., color, render texture, etc.) provided in the virtual environment.

In some embodiments, the XR-NPF system may be bi-directional, and allow the user to control their interaction with the virtual environment based on neural signals. For example, sensors (external, implanted, or combinations thereof) may be used to measure activity, such as electrical signals, in the user's nervous system. The XR-NPF system decodes those signals and determines the user's intended interaction based on the decoded signals. A stimulation pattern may then be generated based on the determined interaction and applied to the user via the neural interface. The stimulation may be useful as feedback to the user to help guide their neural interaction with the virtual environment.

In this manner, the XR-NPF may be used to connect neural stimulation based percepts with interaction with a virtual world. This may be useful for a variety of applications. For example, the percepts may be useful as feedback to user (e.g., analogous to the concept of haptics). For example, if the interaction is touch it may help the user ‘feel’ a percept linked to ‘touching’ a virtual object, which may aid in the immersion of the experience. The XR-NPF may also be useful to help train the user to recognize certain stimulation patterns, e.g., for clinical use. For example, by applying a certain stimulation pattern when the user touches a ‘soft’ object in the virtual environment, the user may come to associate that stimulation pattern with the sensation of softness, which may then be used to help rehabilitate the user (e.g., by applying that stimulation pattern when they touch something soft with a prosthetic in a non-virtual environment in order to give them a sense of touch with the prosthetic). The XR-NPF may also be useful to allow for percepts which it is not possible for a user to experience. For example, an AR system may apply a percept when the user is moving in a correct direction in order to give the user a sense of direction. The XR-NPF may also be useful to allow users whose interaction may be limited in the real world to experience a more immersive virtual environment. For example, a paralyzed user may be able to use a bi-directional XR-NPF to both interact with a virtual environment and receive percepts based on that interaction without the use of their limbs.

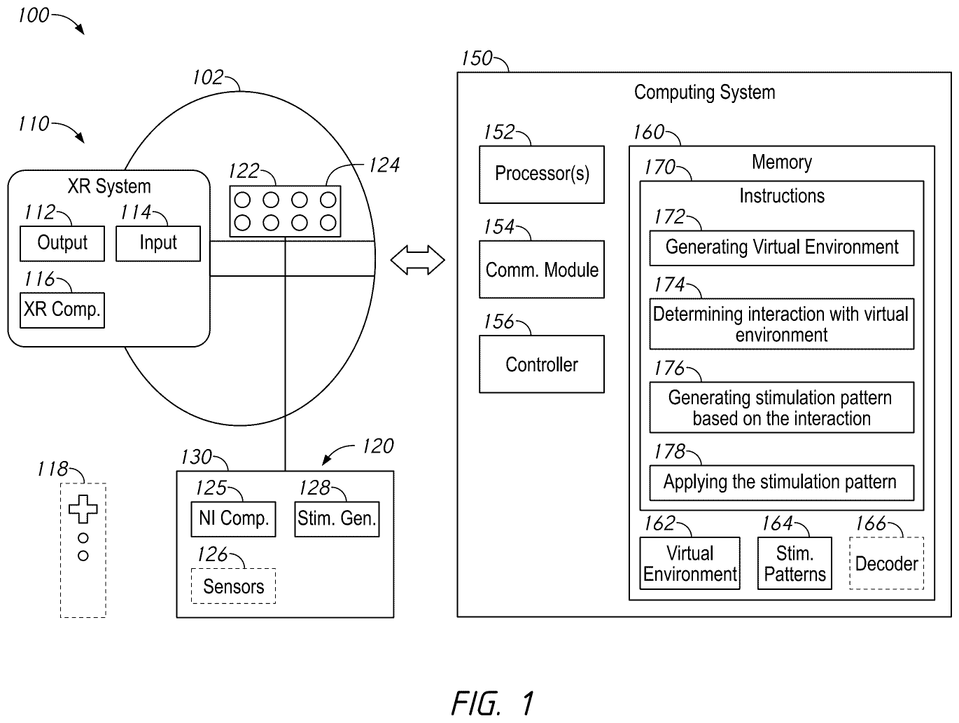

FIG. 1 is a block diagram of an XR-NPF system according to some embodiments of the present disclosure. The XR-NPF system 100 includes an extended reality (XR) system 110 and neural interface 120. The XR system 110 presents a virtual environment to a user 102. The neural interface 120 applies stimulation to a nervous system of the user 102 based on the user's interaction with the virtual environment presented by the XR system 110. The XR-NPF system 100 also includes a computing system 150 which operates the XR system 110 and neural interface 120. Although shown as a separate component, in some embodiments, one or more elements described with respect to the computing system 150 may be distributed in the XR system 110 and/or neural interface 120. In some embodiments, there may be more than one computing system, such as an XR computing system which operates the XR system 110 and a neural interface system which operates the neural interface 120.

The XR system 110 includes one or more outputs 112 which present a virtual environment to the user 102. The XR system also includes an input system 114 which allows the user 102 to interact with the virtual environment. In some embodiments, the XR system 110 may include an optional controller 118, which is operated by the user 102 to provide inputs to the input system 114. Other methods of interaction with the virtual environment may be used in other embodiments. The XR system 110 may also include one or more elements of an XR computing system 116, such as an onboard processor, memory, and so forth, which may help the XR system 110 to operate.

The neural interface 120 includes one or more stimulation devices 122 which can provide stimulation to the user's 102 nervous system, record information from the user's nervous system, or combinations thereof. For example, the stimulation devices 122 may be implantable electrodes. Other stimulation systems may be used in other example embodiments. The neural interface 120 also includes a stimulation generator 128 which applies a stimulation pattern to the stimulation devices 122 based on instructions from the computing system. Similar to the XR system 110, in some embodiments, the neural interface may include a neural interface computing system 125 which includes one or more elements such as a processor, memory, and so forth.

In some embodiments, the neural interface 120 may include one or more sensors 126 which record information from the user 102, such as activity in the user's nervous system. In some embodiments, the implantable electrodes which act as the stimulation devices 122 may also act as the sensors 126. In some embodiments, the neural interface may include one or more implantable components 124 which are coupled to one or more external components 130 (e.g., a belt pack). FIG. 1 shows the stimulation devices 122 as part of an implantable component 124, while the other components of the neural interface 120 are in an external unit 130, however other arrangements are possible in other example embodiments.

The computing system 150 includes one or more processors 152 which execute instructions 170 in a memory 160. The computing system 150 also includes a communications module 154 which allows it to communicate with the XR system 110 and neural interface 120 and a controller 156 which allows it to send and receive signals from the XR system 110 and neural interface 120.

For ease of explanation, FIG. 1 is described with respect to a singular computing system 150 which is separate from the XR system 110 and neural interface 120. However, the functions and components of the computing system 150 may be divided and/or repeated between any number of devices, including the XR system 110 and neural interface 120. For example, in some embodiments, both the XR system 110 and neural interface may have separate computing systems which are in communication with each other. In some embodiments, the functions of the computing system 150 may be included in the XR computing system 116 and neural interface computing system 122 and a separate computing system may not be needed.

The user 102 is an animal which interacts with the XR-NPF system 100. For example, the user 102 may be a mammal. In some embodiments, the user 102 may be a human. In some embodiments, the user 102 may be a patient who is undergoing a treatment. For example, the user 102 may be disabled as a result of a stroke or other condition. In some embodiments, the user 102 may be a research subject. In some embodiments, the user 102 may be able-bodied. In some embodiments, the XR-NPF system 100 may be a part of a treatment plan, a research project, a recreational system, or combinations thereof.

The XR system 110 may be a virtual reality (VR) system, an augmented reality (AR) system, or combinations thereof. A VR system may generally present a self-contained virtual environment which is separate from the real environment the subject 102 is located in. An AR system may generally mix elements of the virtual environments with elements of the real environment (e.g., by presenting an overlay, a ‘heads up display’, a virtual environment based on the geometry detected in the real environment, or combinations thereof). For example, a VR system may generally block out stimulation from the real environment (e.g., by covering the eyes and presenting displays of the virtual environment instead) while an AR system may project images of the real environment overlaid with virtual elements. In some embodiments, the XR system 110 may be a commercial XR system such as an HTC VIVE™, a META QUEST™, or an APPLE VISION PRO™.

The XR system 110 includes one or more output systems 112 for presenting the virtual environment the subject 102. For example, the output systems 112 may include one or more displays configured to present images of the virtual environment, one or more speakers designed to present sounds of the virtual environment, or combinations thereof. In some embodiments, the output systems 112 may include two displays, one positioned to be viewed by each eye of the subject in order to present stereoscopic images. Other display geometries may be used in other embodiments. Similarly, in some embodiments, the output systems 112 may include two speakers each configured to present sound to one of the subject's ears in order to generate stereo sound. Other sound presentation geometries may be used in other example embodiments. In example XR systems 110 with both stereoscopic video and sound, the user 102 may experience a heightened sense of being immersed in the virtual environment. In some embodiments, the XR system 110 may take the form of a headset which when worn by the user 102 places the one or more displays over the user's 102 eyes. For example, the XR system 110 may include a unit configured to fit over the user's face and position the displays over the user's eyes, and one or more straps which hold the unit on the user's head.

The XR system 110 includes one or more input systems 114 which determine actions taken by the user 102 and interpret them into interactions with the virtual environment presented by the output systems 112. The input system 114 may include one or more inertial measurement units which track a position and or orientation of one or more components of the XR system. An example inertial measurement unit may include one or more gyroscopes, accelerometers, or combinations thereof. For example, the input system 114 may include an accelerometer which measures an orientation of the user's head. Based on the measured orientation, the computing system 150 may update the view of the output 112 to match the orientation of the head in order to represent the user ‘looking around’ the virtual environment. In some embodiments, the input system 114 may include a microphone in order to receive voice commands. In some embodiments, the input system 114 may include position tracking of the user's body 102 in an environment (e.g., to match motion in the real environment to motion in the virtual environment). In some embodiments, the input system 114 may include a microphone (e.g., for voice control). In some embodiments, the input system 114 may include components in a headset worn by the user 102, components in separate units, or combinations thereof. For example, in some embodiments, the input system 114 may include one or more controllers, such as a handheld unit 118 with one or more buttons or controls on it. For example, the handheld unit 118 may include a joystick, analog stick, d-pad, one or more buttons, accelerometer, markers for position tracking, or any combination thereof. In some embodiments, the handheld unit(s) may be a commercial controller not specifically designed for use with the XR system 110, such as a controller for a video game system. In some embodiments, the handheld unit(s) may be designed for operation with the XR system 110, such as controllers packaged with a commercial XR headset 110. In some embodiments, the input system 114 may include any combination of input methods described herein.

The neural interface 120 interacts with the user's 102 nervous system to generate percepts based on stimulation patterns generated by the computing system 150. The neural interface 120 may use one or more stimulation devices 122 to apply a stimulation pattern to the user 102. For example, the neural interface 120 may include a stimulation generator 128 which receives the stimulation pattern(s) from the computing system 150. Responsive to the stimulation patterns, the stimulation generator 128 applies a voltage, current, or combination thereof to the stimulation device 122. In some embodiments, the stimulation device may include elements in contact with the user 102. In some embodiments, the stimulation device may be implanted. In some embodiments, the stimulation device 122 may be a non-contact device.

In some example embodiments, the neural interface 120 may include a direct electrical stimulation (DES) system which applies electrical stimulation via implanted electrodes. In some embodiments, the electrodes may be implanted in the user's central nervous system, peripheral nervous system, or combinations thereof. There may be one or more electrodes implanted as part of the implantable unit 124 which act as the stimulation devices 122. The electrodes apply a voltage based on a stimulation pattern applied by the stimulation generator 128 in response to a stimulation pattern provided by the computing system 150. For example, the stimulation pattern may specify how the applied voltage varies over time and the stimulation generator 128 may generate and apply those voltages to the electrodes. In some embodiments, different electrodes may be given different stimulation patterns. In some embodiments, the electrodes may be implanted in regions of the user's 102 nervous system which cause percepts when stimulated. For example, the electrodes may be implanted in a sensory region of the user's brain.

The computing system 150 operates the XR-NPF. The computing system 150 is in communication with the XR system 110 and the neural interface system 120 via a communications module. The computing system 150 may communicate with the XR system 110 and neural interface system 120 via wired communications, wireless communications, or combinations thereof. The computing system 150 includes instructions 170, loaded in a memory 160, such as a non-transitory computer readable medium. The one or more processors 152 execute the instructions 170 to operate the XR-NPF system 100. The memory 160 may also include additional information which may be useful to operating the XR-NPF system, such as a virtual environment, a library of stimulation patterns 164, or an optional decoder 166.

The instructions 170 include block 172, which describes generating a virtual environment. Block 172 may cause the computing system 150 to generate a virtual environment 162. Block 172 may include presenting the virtual environment to the user 102. For example, block 172 may cause the computing system 150 to provide one or more data streams to the XR system 110, which in turn causes the XR system 110 to provide outputs to the user 102 via the output systems 112. For example, the data streams may represent one or more graphic feeds which are presented via one or more displays, one or more audio feeds which are presented via a speaker, or combinations thereof.

In some embodiments, the provided data streams may be based, at least in part, on a current state of the virtual environment 162. For example the virtual environment may include an avatar positioned within the virtual environment 162. In some embodiments, the avatar may represent the user 102 or a digital stand-in thereof. Block 172 may include providing information about the virtual environment based on a position of the avatar within the virtual environment 162. For example, the graphic feed may represent a viewpoint of the avatar within the virtual environment.

In some embodiments, block 172 may be performed on an ongoing basis. For example, the virtual environment 162 may be presented to the user 102 at a rate which simulates movements. Accordingly, the virtual environment 162 may be updated on an ongoing basis and presented on an ongoing basis to simulate an immersive real environment.

The instructions 170 include block 174 which describes determining a user interaction with the virtual environment. For example, the computing system 150 may receive one or more input feeds from the input system 114. The input feeds may include information about where the user is looking (e.g., based on accelerometer data from a headset), data from controls such as voice controls or the controller 118, data from the sensors 126 or combinations thereof. Based on the input feeds, block 174 may cause the virtual environment 162 to be updated, which in turn may update the virtual environment being presented to the user 102. For example, if the input feed indicates that the user 102 has turned their head, in the virtual environment 162 may be updated to reflect a rotation of the avatar. This in turn may lead to updated graphic information (e.g., a new point of view on the virtual environment 162) to be provided to the output system 112.

In some embodiments, as described in more detail herein, the neural interface 120 may include one or more sensors 126 which may measure activity in the user's 102 nervous system. These sensor inputs may be passed to the computing system 150 and treated as if they were inputs from the input system 114 of the XR system 110. This may be particularly useful in situations where the user 102 has limited movement (e.g., paralysis), however, it is not necessary for the user's 102 motion to be limited for sensors 126 to be used. In some embodiments, the sensors 126 may be affixed to various points on the user's body 102 to measure signals (e.g., EEG and/or EMG sensors). In some embodiments, the sensors 126 may be non-contact sensors (e.g., readings from an fMRI). In some embodiments, the sensors 126 may be implantable components 124. For example, along with the stimulation electrodes 122, the implantable components 124 may include electrodes positioned to record information from one or more parts of the subject's 102 nervous system. In some embodiments, the sensors 126 and stimulation devices 122 may be implanted at the same location. In some embodiments, the sensors 126 and electrodes 122 may be implanted at different locations. For example, the sensors 126 may be implanted at a motor region of the brain while the stimulation devices may be implanted at a sensory region of the brain.

The computing system 150 may include an optional decoder 166 which may be used to perform neural signal decoding to interpret the signals from the sensors 126 into an input. For example, if the sensors 126 measure neural activity at a motor or intention forming region of the brain, the decoder 166 may be used to interpret those signals as a movement. This decoded information may be provided as an input feed and used by block 174 to determine the user's interaction with the virtual environment. For example, the sensor information may be decoded into an intention to move a hand, and responsive to that, block 174 may cause the avatar's hand to move in the virtual environment.

Some example applications may involve the user 102 ‘touching’ or ‘grabbing’ virtual objects in the virtual environment. In some embodiments, the input system 114 may provide data that represents movements of an avatar's hands, and grasping of those hands. For example, the input system 114 may include motion tracking of the user's 102 arms and hands in the real environment, and this may be mapped to movement of the avatar's arms and hands. In another example, a thumbstick on the controller 118 may be used to represent movement of the arms, and a button press may indicate grasping. In another example, the sensors 126 may record signals which are decoded into movement of the arm and/or grasping, even if the user 102 is not moving in the real environment. In some example embodiments, combinations of these methods may be used, for example, by using motion tracking to determine a location of the arm/hand, and using a button press on a controller to determine grasping.

The instructions 170 include block 176, which describes generating a stimulation pattern based on the interaction. For example, responsive to the interaction, the computing system 150 may select a stimulation pattern. In some embodiments, the stimulation pattern may be selected from a library of stimulation patterns 164. For example, the library may include predetermined types of stimulation pattern (e.g., sine wave, square wave, sawtooth, etc.), predetermined parameters of stimulation pattern (e.g., certain magnitudes and/or durations of stimulation), or combinations thereof. In some embodiments, block 176 may include generating the stimulation pattern by dynamically generating the stimulation pattern.

Block 176 may include selecting the stimulation pattern based on a type of the interaction. For example, if the interaction is with a first type of object in the virtual environment 162, a first type of stimulation pattern may be generated. If the interaction is with a second type of object in the virtual environment, a second type of stimulation pattern may be generated. In some embodiments, certain types of interaction may be associated with stimulation patterns, while other types of interaction do not cause boxes 176 and 178 to be performed. For example, looking around the virtual environment may not cause stimulation to be applied, while interacting with a virtual object in the virtual environment 162 may cause a stimulation to be applied. In some embodiments, the stimulation pattern may continue to be generated while the interaction is ongoing. For example, if the interaction is ‘touching’ a virtual object, then the stimulation may continue while the virtual object continues to be touched. What types of interactions cause stimulation and what stimulation is generated may be dependent on the application that the XR-NPF system 100 is being used for.

The instructions 170 include box 178, which describes applying the stimulation pattern. Responsive to the instructions in box 178, the computing system 150 provides the stimulation pattern generated in box 176 to the neural interface. In some embodiments, the stimulation pattern may be provided to a stimulation generator 128 of the neural interface 120. Based on the received stimulation pattern, the stimulation generator 128 applies the stimulation via the stimulation device 122. In some embodiments, the computing system 150 may directly provide the stimulation pattern to the stimulation system 122. The stimulation system 122 applies the stimulation pattern to the user's nervous system 102, which can cause a percept in the user 102.

The boxes 172-178 may represent a process which is run continuously on the computing system 150 while the user 102 is using the XR-NPF system 100. For example, as the user 102 continues to interact with the virtual environment 162, the XR-NPF system 100 will continue to generate and apply stimulation patterns based on those interactions. The XR-NPF system 100 may operate in a real-time or pseudo-real time fashion.

In at least some example applications, the XR-NPF system 100 may be used to help a user 102 feel more immersed in a virtual environment. For example, by applying stimulation when certain interactions are performed. This may be analogous to the haptic feedback used in smart devices with touchscreens, where vibration of the device gives the user confidence that they are touching buttons on a purely touch screen. In an analogous fashion, the use of neural-perceptual feedback may give the user a stronger sense that they are interacting with the virtual environment. In some example embodiments, the use of different stimulation patterns for different interactions and/or interactions with different types of thing, may allow for easier recognition of virtual objects. For example, if a first stimulation pattern is applied when the interaction is with a ‘soft’ virtual object, and a second stimulation pattern is applied when the interaction is with a ‘sharp’ virtual object, the user 102 may be able to perceive the difference between types of object via virtual ‘touch’ alone.

In at least some example applications, the XR-NPF system 100 may be used to train a user 102 to experience certain percepts responsive to certain stimulation patterns. For example, by repeatedly applying the same stimulation pattern when a ‘soft’ virtual object is touched in the virtual environment, the user 102 may come to associate that stimulation pattern with touching a soft object. This stimulation pattern may then be applied for real world interactions, for example if the user 102 touches a soft object with a prosthetic.

In at least some example applications, the XR-NPF system 100 may be used to provide the user 102 with percepts they are otherwise incapable of experiencing. For example, the XR-NPF system 100 may allow for a simulation of a perceptual ability which was lost due to disease or injury. For example, a person who no longer has the ability to experience touch because of disease and/or injury may be able to perceive ‘touch’ based on their interactions in the virtual world. In some examples, the XR-NPF system 100 may allow for extended sensory perception beyond what is normally available. This could include delivering stimulation to enable sensory information (i.e. texture, temperature) of nonlocal virtual objects and objects interacted with in ways other than avatar interaction (e.g. gaze ray based object selection and interaction.) As another example, a user wearing an augmented reality system may receive stimulation patterns to provide directions using a sensation which indicates they are currently moving along the proscribed path instead of (or in addition to) a visual stimulus projected on their field of view.

In some embodiments, one or more of the XR system 110, the neural interface 120, and the computing system 150 may be a general purpose systems intended for commercial, clinical, and/or research applications. For example, the computing system 150 may be implemented by one or more general purpose computers, such as laptops, desktops, tablets, smart devices and so forth. The XR system 110 may be a commercial XR system sold for general purposes, such as a VR headset. The neural interface 120 may be a system installed in the user 102 for a purpose other than the XR-NPF system 100. For example, the neural interface 120 may be a clinical stimulation system implanted in the user 102 in order to help manage or treat one or more diseases and/or conditions of the user 102. In some embodiments, the XR-NPF system 100 may be implemented by application system software installed across one or more of the different components in order to allow them to work together as an XR-NPF system.

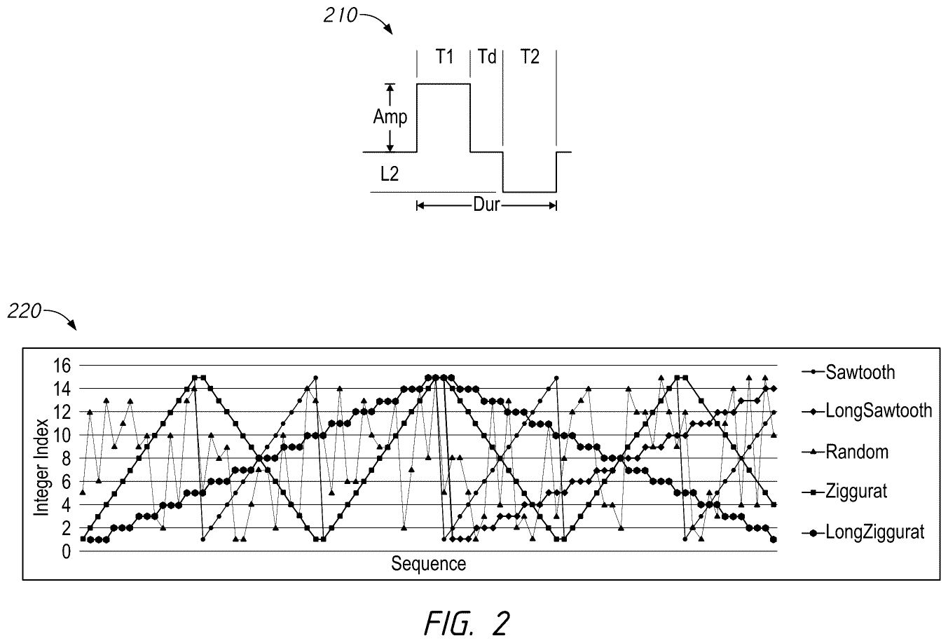

FIG. 2 shows graphs of example stimulation patterns according to some embodiments of the present disclosure. The graphs 210 and 220 may, in some embodiments, represent different stimulation patterns which may be generated and applied by a stimulation system such as the XR-NPF system 100 of FIG. 1. In some embodiments, the graphs 210 and 220 may represent a library of different stimulation patterns which may be applied, such as the stimulation pattern library 164 of FIG. 1.

The graphs 210 and 220 shows an example of amplitude modulated stimulation. Other types of stimulation may be applied in other example embodiments. The graph 210 shows an example biphasic pulse over a pulse duration. The graph 220 shows how the biphasic pulses of graph 210 may be assembled into an amplitude modulated stimulation pattern.

Graph 210 shows a biphasic pulse. The pulse has a duration Dur and an amplitude Amp. The biphasic pulse may represent a voltage applied to a stimulation device (e.g., 122 of FIG. 1) such as a stimulation electrode. For a first period of time T1, the voltage is raised from a previous level, such as a baseline level, to a higher level. The higher level has a voltage difference from the base line based on the amplitude Amp. When the first period of time T1 ends, for a delay time Td, the voltage returns to the baseline level. In some embodiments, the delay time may be nothing. After the delay time, for a second period of time T2, the voltage is driven to a level below the baseline. At the end of the second period of time, a new biphasic pulse may begin.

The graph 220 shows a number of waveforms represented as a stimulation amplitude Amp (vertical axis) for a number of stimulation periods Dur, with each dot along the horizontal axis representing a stimulation period Dur. In other words each dot may represent the biphasic pulse 210 of FIG. 2, but with an amplitude Amp determined by the height along the vertical axis of graph 220. For the sake of description, each of the waveforms has been given a name based on the shape of the waveform. For example, the sawtooth increases from a minimum amplitude to a next higher amplitude with each successive stimulation period until it reaches a maximum amplitude and then it drops down to the minimum again on the next stimulation period. The long sawtooth is similar to the sawtooth except that each time it increases it remains increased for a quantity of stimulation periods (in this example three stimulation periods) before increasing again. The random waveform, randomly selects a stimulation intensity with each stimulation period. The ziggurat increases from a minimum amplitude to a next higher amplitude with each successive stimulation period until it reaches a maximum amplitude, and then it begins decreasing with each successive stimulation period. The long ziggurat is similar except that each time it changes, it remains at that level for a quantity of stimulation periods (in this example 3 stimulation periods) before changing again.

Each waveform of the graph 220 may generate a different percept when applied to the user. By selecting different stimulation patterns based on different interactions, an example XR-NPF system may generate different percepts in the user responsive to different interactions.

FIG. 2 shows only one example way of applying different stimulations. For example, instead of modulating the amplitude, the interpulse latency L2 could be modulated instead. Similarly, while graph 210 shows an example of a basic stimulation waveform, other stimulation waveforms may be used and modified to form stimulation patterns in other example embodiments.

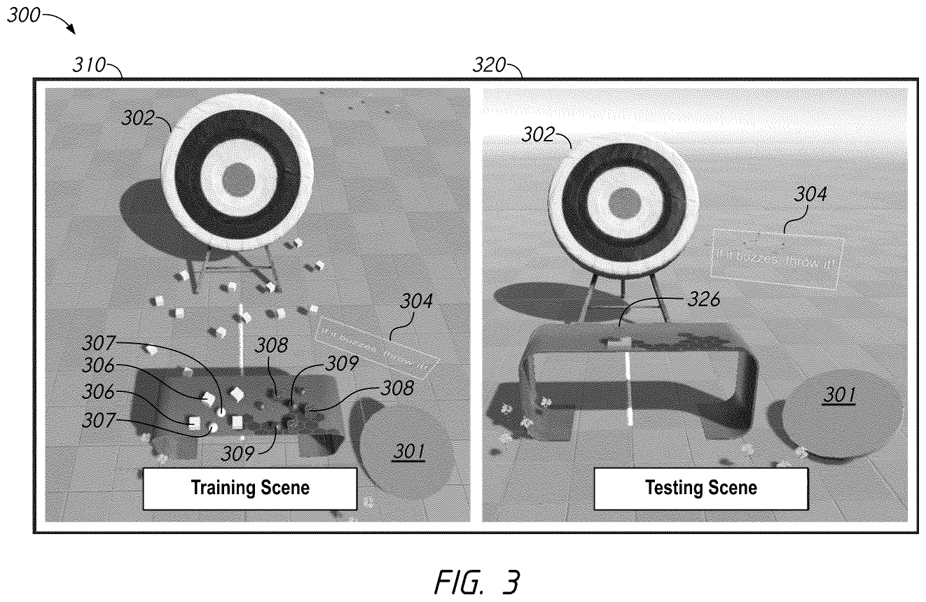

FIG. 3 shows a pair of images which represent views of an example application for binary discrimination according to some embodiments of the present disclosure. FIG. 3 shows example views 310 and 320 of two different modes of an example application 300. The application 300 may be run by an XR-NPF system such as the XR-NPF system 100 of FIG. 1, in some embodiments. The application 300 represents a virtual environment (e.g., 162 of FIG. 1) as well as rules for how stimulation is applied based on different interactions and what stimulation is applied.

FIG. 3 represents an example application 300 used to train a user (e.g., 102 of FIG. 1) to be able to distinguish between types of virtual object based on stimulation applied via a neural interface (e.g., 120 of FIG. 1) when the user interacts with the virtual environment to ‘touch’ the virtual object using an XR system (e.g., 110 of FIG. 1). The application has a training mode 310 and a testing mode 320. In the training mode 310, other information is provided to the user via the virtual environment to help distinguish between the types of object, while in the testing mode 320 only the stimulation is used to distinguish objects.

The images shown in FIG. 3 are used to give examples of a virtual environment presented to a user via an XR system. FIG. 3 shows two example 2D images, one for the training mode 310 and one for the testing mode 320. However, while using the XR-NPF system, the user may be presented with a more immersive view of the virtual environment. For example, the user may be presented with stereoscopic images to show them a 3D environment, and may be able to ‘look around’ by moving their head.

The user may be able to interact with objects in the virtual environment through one or more methods. In the example application 300, the user is capable of interacting by picking up virtual objects, dropping picked up virtual objects, and throwing picked up virtual objects. For example, the user may be able to manipulate a representation of an avatar's hand (not shown in FIG. 3). In some embodiments, the hand may move responsive to motion tracking of the user's arm/hand, motion tracking of a controller, movement of control input device such as a joystick or mouse, or combinations thereof. In some embodiments, the hand may move responsive to a decoder (e.g., 166) interpreting signals from sensors measuring activity of the user's nervous system. When the user moves the hand in proximity to a virtual object, the user may be able to ‘grasp’ the object to pick it up. For example, the grasping may be performed responsive to motion tracking such as of the user closing their hand, depressing a button, or combinations thereof. In some embodiments, the grasping may be performed responsive to a decoder interpreting signals from sensors measuring activity of the user's nervous system. Once grasped the XR-NPF system may attach the object to the representation of hand, and as long as the object remains grasped, the object may generally move wherever the hand moves. This may simulate holding the object.

The user may also be able to let go of a held object, for example by performing the action which grasped the object again or by stopping performing the action which grasped the object. For example, if pressing a button and holding it down grasps an object, then releasing the held button may release the object. The user may be able to either drop or throw the object when they release it. In some embodiments, whether the object is dropped or thrown may be based on a speed the user is moving the avatar's hand when the object is released. For example, if the user is not moving the avatar's hand, the released object may be dropped and may move generally downwards. If the user is moving the avatar's hand, the object may be thrown. In some embodiments, the XR-NPF may implement a physics engine in the virtual environment to simulate qualities like momentum and gravity in order to determine the movement of virtual objects when they are released.

The application 300 may take the form of a game, where the user is presented with an instruction 304, to throw one type of virtual object 306-309 towards a target 302, while dropping the other type of virtual object in a disposal bin 301. The types of object may be distinguished by whether or not a stimulation is applied to the user's nervous system while the object is being grasped. In both modes of the game, when the application 300 begins, the virtual environment includes a virtual table with a set of one or more virtual objects 306-309 or a button 326 on it. A virtual target 302 is presented a distance ‘behind’ the virtual table (relative to the user's viewpoint), and a virtual disposal bin 301 is positioned next to the virtual table. The user is instructed via instruction box 304 to grasp the objects and throw them towards the target 302 if they experience a buzzing sensation. If they do not experience the buzzing sensation, they should drop the virtual object in the bin 301. When a first type of object is grasped, no stimulation is applied. When a second type of object is grasped, the XR-NPF system generates a stimulation pattern and applies it to the user, which the user perceives as a buzz. For example, in some embodiments, the system may apply one or more of the stimulation patterns of FIG. 2.

The virtual objects 306-039 may include one or more virtual objects of each type. For example, during the training mode 310, the table is initialized with a set of objects which include multiple shapes of object, each of which can be the first type of object or the second type of object. For example, the virtual objects may include spheres 307 and 309 and cubes 306 and 308. These include a first type of cube 306 and a second type of cube 308 and a first type of sphere 307 and a second type of sphere 309. During the training mode 310, additional information is presented to the user, for example, the objects of the first type 306 and 307 are rendered with a first color and texture while the objects of the second type 308 and 309 are rendered with a second color and texture. This may allow the user to use senses other than the percept caused by the stimulation to distinguish the type of object. In some embodiments, other feedback may also be used to help the user distinguish between types of object. For example, a controller may vibrate when the second type of object is grasped but not when the first type of object is grasped.

During the testing mode 320, a button 326 is presented on the table. In some embodiments, the button 326 may be interacted with in a manner similar to the way in which the objects are grasped. For example when the user moves the avatar's hand near the button, the interaction which caused grasping may instead activate the button 326. When the button is activated, a test object is generated in the virtual environment.

The test object can only be determined to be of the first or the second type based on whether the user experiences a percept (e.g., a buzzing feeling) from stimulation or not when they grasp the object. If the user is able to correctly determine what to do with the object, it may indicate that they have learned to associate the stimulation with certain interactions in order to experience information (the type of object) which is not otherwise available to them. During both the testing and training modes, a marker of success may be given if the object is correctly tossed at the target 302 or dropped in the bin 301. For example, a score may increase, or feedback may be presented in the virtual world such as sparkles, a noise, etc.

In this manner, the user may test interactions in the testing mode 310 to get a sense for what it feels like when stimulation is applied when they ‘touch’ certain virtual objects. After training, they may proceed to the testing mode 320 where they can demonstrate if they are able to distinguish objects based on the stimulation, which may act in a manner analogous to a sense of touch.

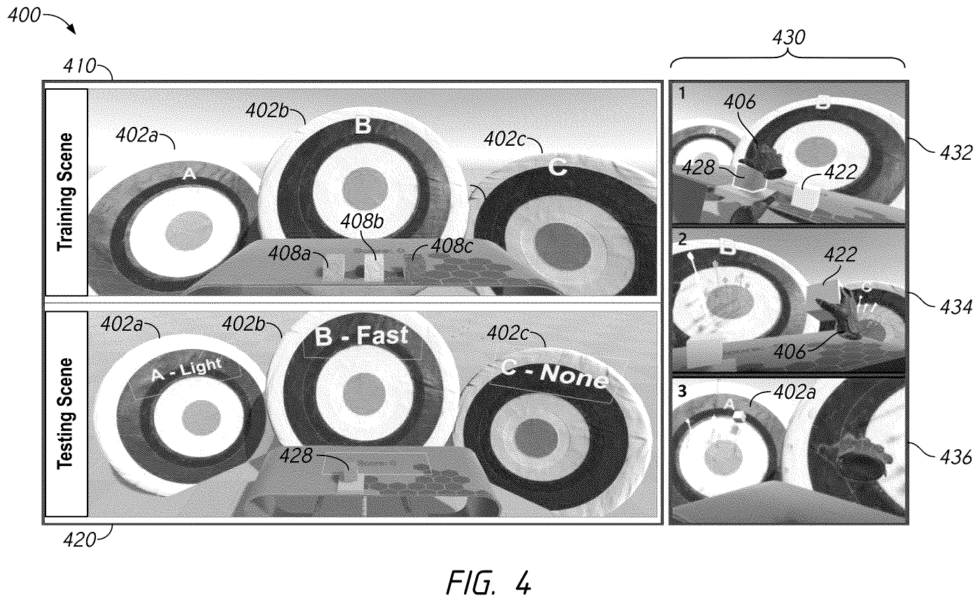

FIG. 4 shows a set of images which represent views of an example application for ternary discrimination according to some embodiments of the present disclosure. FIG. 4 shows example views 410 and 420 of two different modes of an example application 400. FIG. 4 also shows an inset 430 which represents a example sequence of actions. The application 400 may be run by an XR-NPF system such as the XR-NPF system 100 of FIG. 1, in some embodiments. The application 400 represents a virtual environment (e.g., 162 of FIG. 1) as well as rules for how stimulation is applied based on different interactions and what stimulation is applied. The application 400 may share certain similarities with the application 300, for example in terms of how objects are grasped, dropped, and thrown, the goal of throwing objects at a target, and different objects being of different types which determine applied stimulation. For the sake of brevity certain details already described with respect to FIG. 3 will not be repeated again with respect to FIG. 4.

Unlike the application 300 of FIG. 3, in the application 400 of FIG. 4, there are three types of object, shown in the training mode 410 as representative objects 408a, 408b, and 408c respectively. In the application 400, a first stimulation pattern is applied when a first type of object is grasped, a second stimulation pattern is applied when a second type of object is grasped, and no stimulation is applied when the third type of object is grasped. For example, when the first type of object is grasped, the XR-NPF system may apply a first one of the patterns as shown in the graph 220 of FIG. 2, and when the second type of object is grasped the XR-NPF system may apply a second one of the patterns shown in the graph 220 of FIG. 2.

In the application of FIG. 4, the user is playing a game where they are expected to pick up an object 408 and throw it in a corresponding one of targets 402 based on the type of object. For example, the goal is to throw a first type of object 408a into a first target 402a, throw a second type of object 408b into a second target 402b or to throw a third type of object 408c into a third target 408c.

The training mode 410 of the application 400 may work in a generally similar fashion to the training mode 310 of FIG. 3, where the virtual environment includes different virtual objects of different types on the table. The virtual objects may include additional markers, such as rendered color and/or texture, which may help discriminate between the types of object 408. The user may be expected to grab objects 408 off the table and throw them into the correct target 402, with a score keeping track of correct sorting.

During the testing mode 420, similar to the testing mode 320, objects no longer have secondary characteristics to help determine type. Only whether stimulation is provided, and if so, what type, can be used to determine the type of object. In the testing mode 420, there may be a button 428 on the table. The user may interact with the button 428 in a manner generally analogous to the way the user interacts with the objects 408. In some embodiments, the same action used to pick up the object may be used to activate the button.

The inset 430 shows a set of images which represent an example sequence of actions which may be performed as part of the testing mode 420. The images of the inset 430 show the representation of the avatar's hand 406. In a first image 432, the user 406 moves the representation of the hand to the button 428 and activates it. Responsive to activating the button 428, the XR-NPF generates a test virtual object 422 with a random one of the first, second, and third types on the table. The second image 434 represents the user moving the avatar's hand over to pick up the test object 422. While the user is causing the avatar's hand to grasp the object 422, the XR-NPF determines a type of stimulation to apply (e.g., a first type, a second type, or no stimulation) based on the type of object. In the third image 436, the user throws the virtual object at a selected one of the targets 402a, based on the stimulation they are receiving.

In some embodiments, subjective labels may be applied to the targets 402 to represent the type of object which should be thrown at that target. In the example of FIG. 4, target 402a is labeled light, target 402b is labeled fast, and target 402c is labeled none. This may act as a form of reinforcement which may help the user learn to associate the particular stimulation pattern with that descriptor.

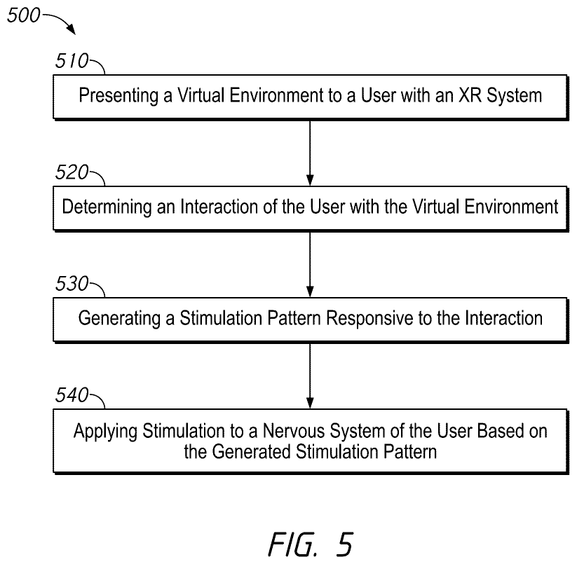

FIG. 5 is a flow chart of a method according to some embodiments of the present disclosure. The method 500 may be implemented by an XR-NPF system. For example, the method 500 may be implemented by the XR-NPF system 100 of FIG. 1.

The method 500 may generally begin with box 510, which describes presenting virtual environment to a user with an XR system (e.g., XR system 110 of FIG. 1). The virtual environment (e.g., 162 of FIG. 1) may be generated by the XR-NPF system. The presenting may include displaying images on one or more displays, playing sound on one or more speakers, or combinations thereof. In some embodiments, the presenting may be an ongoing process, and, for example, the displayed images may be updated at a video rate (e.g., at or above 20 frames per second) to simulate movement.

Box 510 may be generally followed by box 520, which describes determining an interaction of the user with the virtual environment. In some embodiments, the method 500 may include determining the interaction based on motion tracking, controller inputs such as button and joystick operation, voice commands, gesture tracking, or combinations thereof. In some embodiments, the method 500 may include determining the interaction based on receiving information from one or more sensors which measure activity in the user's nervous system and decoding the sensor information into an input. The decoded input may then be used as an interaction with the virtual environment.

An example interaction may include moving an avatar's hand within the virtual environment to place the avatar's hand near a virtual object. The interaction may then include interacting with the virtual object. For example, the example interaction may be performed receiving motion tracking of the controller to track the motion of the user's hand, and then interpreting a button press on the controller as interacting with the object.

Box 520 may generally be followed by box 530 which describes generating a stimulation pattern responsive to the interaction. In some embodiments, the method 500 may include determining whether or not to generate a stimulation pattern based on a type of the interaction. For example, some interactions may be associated with a stimulation pattern while other types of interaction may not be associated with an interaction. In some embodiments, the generating may include selecting a stimulation pattern based on a type of interaction, a type of object being interacted with, or combinations thereof. In some embodiments, the method 500 may include selecting a type of stimulation based on the type of interaction, the type of object being interacted with, or combinations thereof. For example, the method 500 may include selecting one of the stimulation patterns in the graph 220 of FIG. 2. In an example embodiment, the method 500 may include generating a first stimulation pattern responsive to the user causing their avatar to grasp a first type of virtual object in the virtual environment and generating a second stimulation pattern responsive to the user causing their avatar to grasp a second type of virtual object.

Box 530 may be followed by box 540 which describes applying stimulation to a nervous system of the user based on the generated stimulation pattern. For example, the method 500 may include applying voltages to one or more stimulation electrodes. The voltages may vary over time based on the generated stimulation patterns. The method 500 may include applying the stimulation to a perceptual region of the user's nervous system such that they experience a percept while the stimulation is being applied. For example, the method 500 may include applying the stimulation to a sensory region of the user's brain.

In some embodiments, the method 500 may include continuing to apply the stimulation pattern while the interaction is continuing. For example the method may include continuing to apply the stimulation based on the stimulation pattern while the user continues to have their avatar hold a virtual object.

### Example 1

An example study using an XR-NPF system, such as the XR-NPF system 100 of FIG. 1, is described. The example study represents a specific implementation of the XR-NPF system using the example applications of FIGS. 3 and 4 and the example stimulation patterns described in FIG. 2. In Example 1, a VR system was used as the XR system (e.g., 110 of FIG. 1), and the stimulation was applied to an S1 area of the user's brain using direct electrical stimulation (S1-DES) of the user's brain via implanted electrodes. The example application of Example 1 involves ‘neurohaptic’ feedback, where stimulation is used to simulate a sense of touch of a virtual object.

The recruitable population (e.g., for the user 102 of FIG. 1) included patients to be implanted with sEEG and ECOG intracranial electrodes (e.g., as the implantable unit 124 of FIG. 1) for epileptic seizure localization. Direct stimulation of neural tissue was used within known standards of charge density safety and at electrical current magnitudes below 15 mA. Collected data included continuous neural signals recorded from intracranial electrodes, synchronized in-room sound and video, and behavioral time series including kinematic vectors and object interaction flags from within the XR task. Subjective responses to S1-DES and the VR environment were acquired by questionnaire and free response.

Data were collected from two in-patient human subjects following formal consent of the subject and their legal guardian if under 18 years of age. Subject 1 was a 15-year-old male implanted with ECoG and sEEG electrodes, and Subject 2 was a 19-year-old male with SEEG electrodes only. The maximum S1-DES stimulation amplitudes were 6.5 mA in Subject 1 and 5 mA in Subject 2. Also, the left hand of Subject 2 presented with partial motor paralysis attributed to prior neural trauma but intact sensation to both firm and light touch.

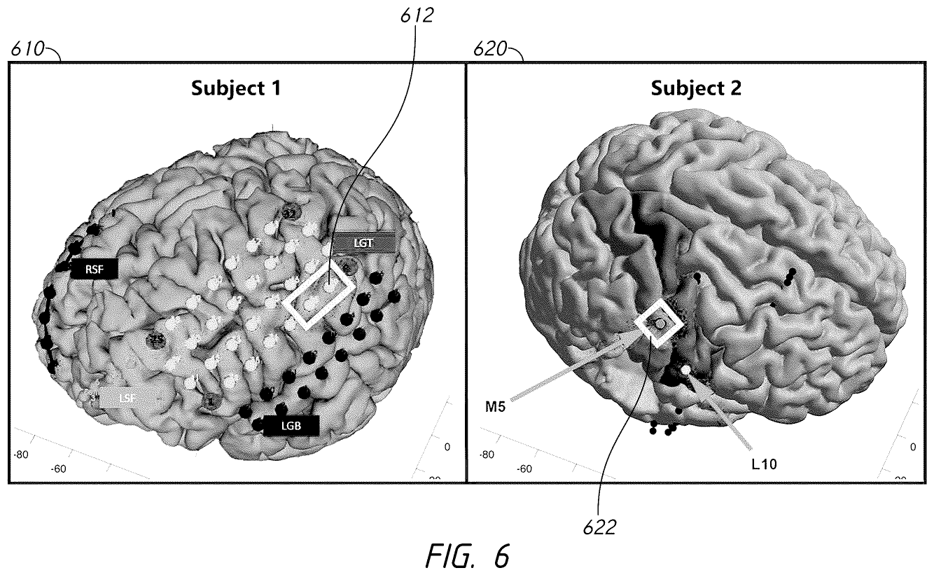

FIG. 6 is a diagram of example electrode localizations for each subject according to some embodiments of the present disclosure. FIG. 6 shows a pair of images 610 and 620, which represent how electrodes were placed on the first subject's brain (image 610) and the second subject's brain (image 620). In example 1, both users were patients who already had an array of implanted electrodes as part of a stimulation system (e.g., 120 of FIG. 1). Imaging of the electrode locations was performed to identify candidate electrodes of the implanted electrodes for use as part of the stimulation for an XR-NPF system (e.g., 100 of FIG. 1).

Prior to stimulation, electrode localization was performed to identify electrodes with likely S1 somatosensory cortex coverage, particularly hand S1 coverage. Post-operative localization was accomplished using preoperative Magnetic Resonance Imaging (MRI) data, and postoperative Computerized Tomography (CT) scans. The post-operative CT scans were co-registered with preoperative T1 MRI using an affine registration through a statistical software package. Three dimensional reconstructions of the pial surface were generated, and electrode channel positions were estimated from postoperative CT. These were then projected onto the reconstructed pial surface (ECOG) or localized subcortically (sEEG). The images 610 and 620 show the three dimensional reconstructions and the electrode positions superimposed as dots. A secondary registration into MNI152 1 mm space enabled automated atlas labelling which aided identification of electrodes implanted on, in, or near the S1 somatosensory cortex. Identification of hand S1 was completed manually.

From these, candidate electrodes were manually identified for direct cortical stimulation of the hand area of the somatosensory cortex (S1-DES). The actual electrodes used per subject are outlined by boxes 612 and 622 respectively. In Subject 1 (image 610), S1-DES between cortical ECOG electrodes in the hand area of the left somatosensory cortex elicited a somatic percept in the right thumb and index filter. In Subject 2 (image 620), S1-DES at cortical sEEG electrodes localized to the hand area of the right somatosensory cortex elicited a sensorimotor percept of clenching and movement across the left hand which had been paralyzed by previous neural trauma.

For Subject 1, ECoG electrodes over the left somatosensory cortex hand (LGT 15:16) and lower arm (LGT 23:24) areas were identified as potential targets for percept-eliciting stimulation. For Subject 2, electrodes were identified in the right somatosensory cortex for hand (4:6), motor cortex for face and tongue (L 8:10), and hand (I 14:15).

A TUCKER DAVIS TECHNOLOGIES™ or TDT system was used for research-grade neural recording and control of direct neural stimulation. A custom control circuit to manage recording, stimulation, and rapid switching between recording and stimulation hardware states was designed. Neural signals were recorded by the TDT PZ5 and RZ2 at 12.2 kHz, with the IZ2 delivering voltage-regulated, constant-current biphasic stimulation pulses in response to real-time VR task dynamics, communicated over a UDP interface between the VR laptop and the RZ2.

Specifically, for task-responsive stimulation, the RZ-2 received UDP packets of single integers between 1 and 15 from the laptop (e.g., computing system 150 of FIG. 1) hosting the XR-NPF application (e.g., in memory 170 of FIG. 1). The arrival of each integer by UDP was used to trigger a single stimulation pulse at an amplitude indexed by the value of the integer. Sending the indices of a 15-element array over UDP instead of amplitude values enabled flexible but structured design of stimulation sequences while also ensuring safe and relevant stimulation magnitudes. This amplitude array was customized for each patient before each experimental session. For example, the patient-specific amplitude array for Subject 1 Session 1 included amplitudes between 3.9 mA and 6 mA in 150 μA steps. For this session, receipt of an integer packet of value 1 would trigger a single pulse of stimulation at an amplitude of 3.9 mA. This design facilitated flexible and safe control of amplitude-modulated sequences responsive to the VR environment.

In parallel with the TDT neural recording system for research neural signal acquisition, clinical neural signals from all implanted electrodes were recorded at a 1024 Hz sampling rate. By hospital protocols, individual contacts were referenced to a scalp needle electrode and grounded to a screw that physically anchored an ECoG or sEEG implant. In-room video was captured at 30 Hz from two orthogonal views of the patient bed, and audio from a wall-mounted microphone. These data were recorded for clinical evaluation of seizure activity. For research, an additional audio synchronization input was used to align VR experimental data with clinical neural data.

Prior to the first experimental session, candidate electrodes identified by the electrode localization process (e.g., as shown in FIG. 6) were evaluated for percept reliability and threshold. Threshold values were approximated using a manual search of candidate electrodes with stimulation values between 1 mA and 6.5 mA, with pulse counts of 1, 5 or 20 pulses at 30 Hz. The presence, strength, location, and character of any evoked percept was determined by subjective report. The perceptual thresholds reported here are approximate, as they were assessed by verbal response and not psychometric curve. Finally, if multiple electrode pairs were found to evoke reliable percepts, the pair with the lowest perceptual threshold was selected.

Following electrode selection, a range of perceivable stimulation amplitudes was identified. As mentioned, this range was identified for each subject at the start of each experimental session. The lower bound of the amplitude range was set at the perceptual threshold of a burst of 5 pulses at 30 Hz, and the upper bound at a consistent and comfortable single-pulse perceptual threshold. In this study, both subjects could reliably detect a single pulse stimulation.

Prior to each ternary discrimination task, amplitude-modulated stimulation sequences were tested for subjective response and discriminability. Up to two seconds of each sequence were delivered and initial subjective descriptions collected. Manual 1-back comparisons between sequences with distinct subjective descriptions were completed until a pair of sequences with strong, untrained discriminability was identified. These two sequences were then selected for use in the ternary neurohaptic discrimination task.

Sequences of indices were used to deliver structured trains of constant-current, charge-matched, biphasic square wave pulses that ranged across a predetermined 15-element array of amplitudes. The amplitude array was customized to the perceptual thresholds of S1-DES for each subject before each session of neuropathic experimentation.

During neurohaptic feedback, S1-DES was delivered as a continuous sequence of constant-current, biphasic, charge-matched, square-wave pulses, such as is shown in the graph 210 of FIG. 2. Pulse width was held constant and is defined as the duration of the entire biphasic pulse. Pulse amplitude is the magnitude of the current across the electrode surface (e.g., Amp of FIG. 2) and was held constant during both phases of a biphasic pulse to ensure the two phases were charge matched.

For amplitude modulated stimulation, five structured sequences of index values were designed and nicknamed based on shape: Sawtooth, LongSawtooth, Random, Ziggurat, LongZiggurat, such as is shown in the graph 220 of FIG. 2. The Sawtooth and LongSawtooth sequences rise from the lowest index or stimulation value (index 1) to the highest (index 15), and then cycle again from index 1 to 15, 1 to 15, etc. LongSawtooth spends three “time steps” at every index for Sawtooth's one. The Ziggurat and LongZiggurat sequences rise from index 1 to 15 and then slowly and symmetrically decrease back to 1. As before, LongZiggurat spends 3 “time steps” at every index for Ziggurat's one. The Random sequence of indices was generated once and was the same sequence in both subjects. The Random sequence may have a much faster oscillatory structure than any of the constructed sequences.

In the embodiment of Example 1, an ALIENWARE™ laptop was used as the computing system (e.g., 150 of FIG. 1), and the HTC VIVE PRO EYE™ head mounted display (HMD) was used as the VR system (e.g., which may implement XR system 110 of FIG. 1 in this example). The example VR headset uses two base stations for submillimeter motion tracking of the handheld controllers (e.g., 118 of FIG. 1) and the headset along 3-euclidian and 3-rotational axes (6D). The base stations were mounted on extensible tripods and positioned a few feet from the user's position to ensure coverage of all potential dynamic movements during VR tasks.

Prior to VR immersion, subjects were introduced to the trigger, grip, and trackpad inputs of the controllers, as well as the fit adjustment mechanisms of the headset (dial, rachet, and strap). The donning and doffing procedures used to position the headset over the headwrap were also discussed prior to first headset placement. Once placed on the head, subjects were guided to adjust the fit of their own headset and encouraged to make the HMD as snug and comfortable as possible. To further reduce the possibility of virtual reality sickness (VRS), our tasks were designed to have simple, neutral backdrops with infinite horizons, few moving objects, and minimal head movement. All tasks were designed using commercial video game development tools such as the UNITY™ game engine.

During active grasp of a neurohaptic object, stimulation indices were sent continuously from the VR environment, over UDP, to the TDT hardware at a rate no faster than 50 Hz. This maximum pulse frequency was set by a delay counter of “physics updates” in the game engine which are 20 ms each.

The two discrimination tasks of this study were built around a throw-to-target game mechanic in which points were earned for successfully hitting the correct target with the correct object. The application 300 of FIG. 3 and 400 of FIG. 4 were used as the discrimination tasks in this example.

HapticSort (e.g., which may implement the application 300 of FIG. 3) is a simple binary (yes/no) discrimination task presented in two scenes: a training scene and a testing scene (e.g., 310 and 320 of FIG. 3). In the training scene, a subject is placed before a table with 13 objects that are all approximately within an arm's reach. As these objects do not regenerate after being thrown, the training scene is limited to 13 trials. Beyond the table is a large, ringed archery target with a green bullseye.

Objects on the table are intermixed spheres (six) and cubes (seven), each rendered with either a matte white material (six) or a metallic gold-orange material (seven) (e.g., objects 306-309 of FIG. 3). The materials indicate whether neurohaptic feedback will (gold-orange) or will not (matte white) be delivered while grasping and holding the object. Subjects are encouraged to determine the neurohaptic state of the object quickly but accurately, and to throw a neurohaptic object towards the bullseye target (e.g., 302 of FIG. 3) and drop a non-haptic object onto a matte brown target (e.g., 301 of FIG. 3). If the subject correctly identifies the neurohaptic object and hits the target, then upon target collision the neurohaptic object explodes into a colorful archery arrow that remains permanently lodged in the target and adopts the color of the ring hit (e.g., green for bullseye). This serves as a visual indicator of success and game score. The actual game score is also projected numerically onto the task table. Behavioral data regarding accuracy of neurohaptic identification was recorded by a script running within the HapticSort scene and confirmed by review of both in-room video and recordings of the VR screen overlay.

After the training scene, the testing scene (e.g., 320 of FIG. 3) is launched. In this scene, subjects are placed before the same table and targets, with the same instruction to throw neurohaptic objects toward the bullseye target and drop non-haptic objects onto the matte brown target. Instead of pre-placed objects on the table, there is a button that, when depressed, generates an object to the right of the button. The location of object generation can be modified easily to accommodate left-hand dominance or various task performance challenges.

Generated objects are pulled from a pre-allocated pool of neurohaptic and non-neurohaptic objects. This is done to reduce memory management of game-instantiated objects and to facilitate even distributions of randomly selected neurohaptic and non-neurohaptic objects. All objects in the testing scene are rendered identically as small, matte white cubes so no visual or VR perceptual characteristic could distinguish neurohaptic verses non-neurohaptic objects, except the delivery of neurohaptic feedback upon contact. Behavioral accuracy was recorded, as before.

To introduce the HapticSort task and validate game design, subjects were asked to first complete the binary discrimination task with traditional vibrotactile haptic feedback, delivered through the VIVE™ controller. Once then HapticSort task was understood, the vibrotactile haptic feedback for object interaction was disabled and only neurohaptic feedback by S1-DES was delivered.

Example 1 also includes a ternary discrimination task such as the application 400 of FIG. 4. HapticSort_ABØ is a ternary discrimination task with visual encoding of S1-DES neuropathic feedback during training and no visual cuing during testing. In this task, subjects perform discrimination between three objects, two with distinct neuropathic profiles and without any feedback in order to throw each type of object onto its own bullseye target.

HapticSort_ABØ is an extension of the binary HapticSort task and demands discrimination between two distinct stimulation sequences in addition to a ‘null’ or no stimulation state. In the training scene of this task, the subject is again placed before a table, but now faces three bullseye targets with visible, in-game labels of A, B, and C (e.g., targets 402a-c of FIG. 4). Placed on the table are three cubes (e.g., 408a-c of FIG. 4), rendered with distinct materials: (left) a blue, translucent, ice-like material, (middle) an opaque, yellow-gold metallic material, or (right) an opaque, orange-red metallic material. These were nicknamed “ice, rock, and lava” by Subject 1.

The goal of the training scene is for the subject to learn which stimulation condition is mapped to which material and which target. The far left “ice” cubes are non-neurohaptic objects, and their correct target is C (Ice→Ø, C). The middle, yellow-gold “rock” cubes elicit one stimulation sequence (e.g., Ziggurat) and their target is A (Rock→A). The rightmost orange red “lava” cubes elicit a second stimulation sequence (e.g., Sawtooth) and go with target B (Lava→B). Once thrown, each cube will regenerate, so the training scene for HapticSort_ABØ is not limited to a preset trial count. Visually distinct materials were introduced to facilitate performance on the training task by providing a visual cue to identify the correct target. This is may be useful for subject engagement early in the task when the stimulation sequences may be difficult to distinguish and remember.

As before, if an object hits its correct target, an archery arrow prefab colored by where it lands on the target is generated, with fanfare. If an object contacts an incorrect target or the floor, it bounces off and does not generate an archery arrow. Once contact with any target or the floor has been made, cubes are returned to their original position on the tabletop after a 3 second delay. The number of trials completed during the training scene of HapticSort_ABØ is determined by the subject, though a minimum of fifteen trials is encouraged.

In the testing scene (e.g., 420 of FIG. 4) of HapticSort_ABØ, the same matte brown button from HapticSort replaces the three, visually distinct cube objects. Upon button press, a matte white cube object appears to the right of the button, as in HapticSort. Cubes are again selected randomly from a pre-instantiated pool of haptic (two of each A and B) and nonhaptic (one Ø) objects. Based on learned target-haptic associations, subjects throw each cube towards the target indicated by its neurohaptic profile.

In Subject 1, although subjective descriptions of the stimulation profiles were elicited, these descriptions were not incorporated into the testing scene. This led to memory errors that decreased behavioral performance and confounded interpretation of target accuracy as an indicator of only perceptual discrimination of the stimulation sequences. In Subject 2, to remove the behavioral confound of needing to memorize target representations (e.g., Stimulation Ø→Target C), one-word descriptors, chosen by Subject 2, were added to the target labels in the testing scene (FIG. 5, left bottom). For example, Target B read “B—Fast” for during one testing scene.

Table 1 summarizes the discrimination accuracy (%) results for both subjects, on all days. Details of the stimulation parameters and sequences are presented in the first, leftmost, column and results for binary stimulation discrimination (‘Stim Binary’ AB-Ø), ternary discrimination (‘Percept Ternary’ A-B-Ø), and sequence discrimination (‘Percept Binary’ A-B) are listed in subsequent columns. Training scene results are indicated by gray backfill.

TABLE I
PERCENT ACCURACY (%) OF HAPTICSORT TRIALS

| | | | |
|---|---|---|---|
| | Stim | Percept | Percept |
| | Binary | Ternary | Binary |
| Stimulation Details | AB - Ø | A - B - Ø | A - B |

Subject 1

| | | | |
|---|---|---|---|
| 13 trials, 200 μs, 3 mA ^{a} | 88.1 | — | — |
| 28 trials, 200 μs, 3 mA ^{b} | 92.9 | — | — |
| 3 trials, 200 μs, 2 mA ^{b} | 0 | — | — |
| 48 trials, 150 μs, 3 mA ^{b} | 95.8 | — | — |
| 52 trials, 400 μs, 4-6.1 mA ^{b} | 100 | — | — |
| LongSawtooth | | | |
| 32 trials, 400 μs, 4-6.1 mA ^{a} | 86.2 | 84.4 | 87 ^{of 23} |
| Sawtooth - LongSawtooth | | | |
| 20, 400 μs, 4-6.1 mA ^{b} | 90 | 50 | 57.1 ^{of 14} |
| Sawtooth - LongSawtooth | | | |
| 46 trials, 400 μs, 4-6.1 mA ^{b} | 86.9 | 73.9 | 77.8 ^{of 27} |
| LongSawtooth - 3.85 mA | | | |

Subject 2

| | | | |
|---|---|---|---|
| 6 trials, 400 μs, 2-3.4 mA ^{a} | 100 | 100 | 100 ^{of 5} |
| Sawtooth - Ziggurat | | | |
| 16 trials, 400 μs, 2-3.4 mA ^{b} | 81.3 | 68.8 | 80 ^{of 10} |
| Sawtooth - Ziggurat | | | |
| 29 trials, 400 μs, 2-3.4 mA ^{b} | 89.7 | 72.4 | 79.2 ^{of 24} |
| Sawtooth - Ziggurat | | | |
| 50 trials, 400 μs, 2-3.4 mA ^{b} | 98 | 82 | 79.5 ^{of 39} |
| Sawtooth - 2 Hz Burst | | | |

^{a} Training scene: visual encoding of objects.
^{b} Testing scene: no visual encoding.

Subject 1 completed three non-consecutive days of neurohaptic VR experiments in which thumb and index finger percepts were elicited by stimulation between contacts LGT 15 and LGT 16 (FIG. 2). The percept was stable in location across experimental days.

Initial percept evaluation with biphasic pulses of 200 us pulse width resulted in the selection of elicited percepts in an amplitude range of 2 mA-4.5 mA.

Prior to neurohaptic delivery, HapticSort familiarization and task validation was completed using controller-based vibrotactile haptics. In the testing scene, controller-based haptics yielded a binary stimulation accuracy, or AB-Ø, of 100%, indicating task comprehension. Vibrotactile feedback was then disabled and multiple rounds of HapticSort with S1-DES were completed.

The discrimination accuracy in the initial HapticSort training scene yielded only an AB-Ø accuracy of 88.1% (Table 1, row 1—gray), despite the visual encoding of the objects in the training scene. During this scene, the subject described the evoked percept from constant amplitude S1-DES as it “may be buzzing.” We note that this response may have been influenced by both the preceding controller-based vibrational haptics and the use of the word “buzz” in the task description.

In the second round of HapticSort (Table 1, row 2), three trials were completed in which the delivered stimulation was subthreshold. Subthreshold stimulation was undetectable by the subject, resulting in a 0% AB-Ø accuracy over the 3 trials.

One week after session 1, biphasic pulses of 400 us pulse width elicited percepts between 4 mA-6.1 mA. Subject 1 was unable to recall if the subjective nature of the short-burst stimulation percept had changed from a week prior and subjective description was difficult to elicit. In contrast, the Sawtooth pattern was described as having a “pulsing effect” with a quick, ˜2 Hz rhythm indicated by a pulsing hand movement. The LongSawtooth and LongZiggurat sequences were both described as “bumpy” and with “different bumpiness.” Specifically, the bumps of the LongSawtooth sequence were described as “sharper.” In contrast, the Random sequence was described as “smoother” and the constant amplitude stimulation train as “flatter.”

After stimulation threshold evaluation and sequence description, Subject 1 completed two rounds of a malfunctioning ternary discrimination HapticSort_ABØ task that encoded a LongSawtooth stimulation object and two null stimulation objects when the second sequence failed to actuate. Considering this a form of binary HapticSort with extra steps, then, the binary discrimination AB-Ø accuracy was 100% over 52 trials (17 neurohaptic and 35 non-haptic, Table 1, row 5).

Four days after session 2, Subject 1 completed three fully functioning rounds of HapticSort_ABØ. The training round and first testing round contrasted null stimulation, Sawtooth, and LongSawtooth sequences using the 4 mA to 6.1 mA percept threshold range, stable from before. LongSawtooth was described as “slow” and Sawtooth as “fast.”

While binary stimulation AB-Ø accuracy was high at 90% over 27 trials of HapticSort_ABØ, binary percept A-B discrimination accuracy was low at only 57.1% over the 14 specific stimulation trials (Table 1, row 7). This low binary percept A-B accuracy was due in part to the confound of needing to memorize associations between sequence percept and target: multiple discrimination errors were made not because A and B were indistinguishable, but because remembering target assignment was difficult. As discussed, this confound was mitigated in Subject 2 by labelling targets with a subjective description of the associated stimulation sequence as well as the A, B, and C designators.

In the second round of HapticSort_ABØ, a constant amplitude stimulation at 3.85 mA was contrasted against the LongSawtooth sequence and null stimulation (Table 1, row 8). LongSawtooth was described again as “bumpy” and the constant amplitude stimulation as “smooth, no bumps.” The target assignment of LongSawtooth was consistent with the first round, preserving the learned association, and the sequences seemed to be easier to discriminate, improving the binary percept A-B discrimination accuracy to 77.8% over 27 stimulation trials.

Subject 2 also completed three days of neural stimulation and neurohaptic VR experiments. On all days, stimulation across right hemispheric cortical sEEG contacts M5 and M6 elicited somatic percepts in the subject's left hand, which was partially paralyzed but retained natural touch perception.

Initial percept evaluation with biphasic pulses of 400 us pulse width elicited percepts in an amplitude range of 2 mA-3 mA.

Upon further evaluation, it was found that a burst of twenty pulses at 2 mA elicited a phantom motor sensation across the palm of the subject's paralyzed left hand, but no discernible or palpable muscle activation. Subject 2 described the evoked percept, saying “I felt like I was clenching” while opening and closing his right hand in a gripping gesture with a regular rhythm. The “phantom motor” percept was also elicited with 10 and 5-count bursts. Again, no actual movement or muscle activation was visible or palpable, just the verbal description of a sensation of movement. An increase in stimulation amplitude to 4.5 mA did not change the percept of movement, although inverse polarity of the biphasic pulse (−4.5 mA) reportedly created a “more intense” feeling of movement or clenching. Reversed contact polarity is equivalent to a negative stimulation current. The sensation of movement was also elicited by single pulse stimulations at −4.5 mA and by 5-pulse burst stimulation at −1 A.

The Sawtooth stimulation sequence across this stimulation range was described as having an irregular “pulsing” rhythm, the LongSawtooth was clearly felt as “a little faster,” and the Random pattern evoked lingering perceptual effects that were initially concerning as evidence of after-discharge activity. The percept elicited by the Random sequence seemed to persist with decaying intensity for more than 3 seconds after cessation of S1-DES. As a result, no further data was collected during this session, and stimulation was suspended for one day to confirm by neural signals visualization and consensus of the clinical team that the Random sequence had not evoked irregular or epileptiform neural activity. Upon review, no unusual neural activity was seen.

Returning to VR neurohaptic tasks a day and a half later, a stimulation range of 2 mA to 3.4 mA was found sufficient to evoke percepts from short burst through single pulse stimulation. On this day, the LongZiggurat sequence was barely perceptible with no distinct characterization, the Ziggurat sequence was perceived as a regular “bump, bump, bump, bump” percept, Sawtooth felt “faster” than Ziggurat, and LongSawtooth was felt as “pulsing” but was “not clearly different” from Sawtooth. Random, cautiously evaluated, was perceived as “just fast pulsing” with no lingering percept.

Subject 2 completed HapticSort familiarization and validation using controller-based haptic feedback. Then, due to time limits, one training and one testing round of HapticSort_ABØ were completed, in which null, Sawtooth, and Ziggurat sequences were contrasted. During training, visually encoded ternary discrimination was performed with perfect accuracy (Table 1, row 9).

On the third day, two additional rounds of HapticSort_ABØ were completed using the same stimulation range as before (2 mA to 3.4 mA). In the first round, Sawtooth, Ziggurat, null stimulations were again contrasted. In the second round, Ziggurat was contrasted with a new stimulation sequence in which a 5-pulse burst of stimulation at 50 Hz was repeated approximately twice per second for a 2 Hz burst frequency. The approximation in frequency is due to slight variability in the timing of VR game updates. This burst stimulation sequence was implemented to more closely replicate the burst frequency approach outlined in the non-human primate study discussed earlier. Overall, Subject 2 performed neurohaptic discrimination with high binary and ternary accuracies (Table 1).

In one example modification of the XR-DES, a multiplier was added to the release velocity of a thrown object, allowing our subjects to more successfully engage in the “throwing” mechanic of the task. The location of various game objects was also adjusted to accommodate the body position and needs of each subject. XR-DES may open opportunities for flexible, creative, and adaptive experimental design not possible except in virtual reality.

### Example 2

An XR-NPF system, such as the XR-NPF system 100 of FIG. 1 may include a bidirectional neural interface (e.g., as the neural interface 120 of FIG. 1). A bidirectional interface uses sensors (e.g., 126 of FIG. 1) to measure activity in one or more locations in the user's nervous system. Based on that measured activity, interaction with the virtual environment is determined and stimulation is applied. In this manner, the XR-NPF system 100 may operate in a manner that minimizes, or even eliminates the need for physical action on the part of the user. This may be especially useful in situations where the user has reduced capabilities, for example due to a stroke or other condition/disability. However, a bidirectional interface may also be useful for those who able-bodied.

Example 2 is described as an extension of Example 1, where a bidirectional interface was added to the example implementation already described with respect to Example 1. Similarly, Example 2 may generally be described with respect to the same Subject 1 and Subject 2 as were described in Example 1.

Example embodiments for bidirectional BCI may include: Transforming a continuous neural activity into one or more logical units, updating to a XR scene, and delivering a stimulation in response to the XR scene. In one embodiment, transforming the continuous neural activity into logical units can include or otherwise comprise real-time bandpower below and above a threshold value, which are encoded as 0 and 1, respectively. In one embodiment, updating to the XR scene can include or otherwise comprise neural inputs directing updates to avatar position, avatar posture, scene lighting, or an object's color. In one embodiment, delivering the stimulation in response to the XR scene can include or otherwise comprise delivering one or more electrical stimulation pulses to the somatosensory cortex during virtual contact (or virtual “collision”) between one virtual object and another virtual object.

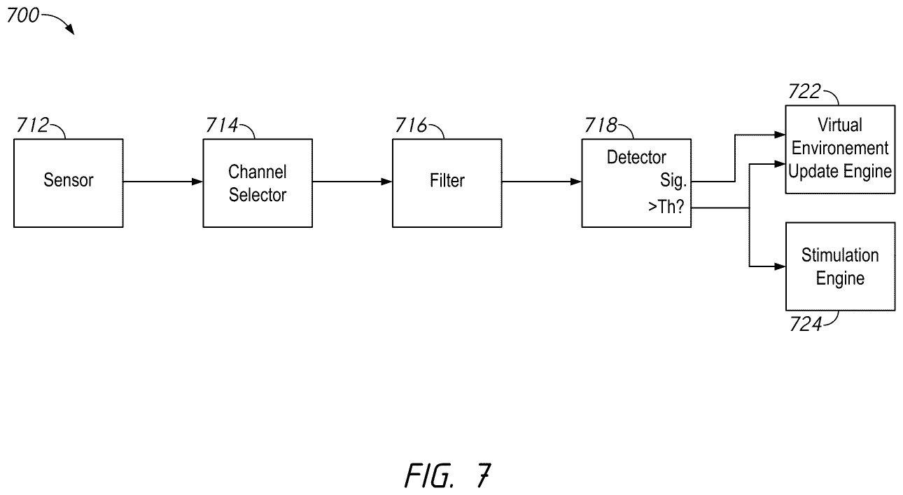

FIG. 7 is an example view of a decoder according to some embodiments of the present disclosure. The decoder 700 of FIG. 7 may, in some embodiments, may be used to interpret signals from a sensor into signals which may be used as inputs for interaction with virtual environment. The decoder 700 is represented as an image of a virtual circuit which represent the operation of the decoder 700. In some embodiments, the decoder may be implemented using similar hardware and software to those described with respect to Example 1, such as using a TDT system for neural recording and stimulation.

The decoder includes a sensor source 712 which receives one or more signals from a sensor coupled to the user's nervous system, a channel selector 714 which selects one or more of the sensor channels, a filter 716 which processes the selected sensor channel, and a detector 718 which determines if the processed selected sensor has crossed a threshold. The detector 718 provides information about if the threshold has been crossed to a virtual environment update engine 722 and to a stimulation engine 724. The sensor source 712 may record information from one or more sensors implanted in the user. Each sensor may act as a separate channel. The selector 714 selects one of those channels and provides it to the filter 716. The filter filters the selected channel. For example, the filter 716 may perform real-time filter-Hilbert processing, producing a continuous bandpower waveform. The detector 718 may represent a virtual oscilloscope or other waveform analysis tool that may be used to visualize, transform, and threshold the selected sensor signal. A binary signal trigger is set based on whether the selected sensor signal has crossed the threshold (e.g., >Th?). If the sensor signal has crossed the threshold, the signal trigger may be a logical high. If the sensor signal has not crossed the threshold, the signal trigger may be a logical low. The virtual environment update engine 722 may update the virtual environment based on a detected interaction if the signal trigger is a logical high. For example, responsive to the signal trigger being high, the virtual environment update engine 722 may cause an avatar's hand to grasp an object. The virtual environment update engine 722 may also receive the signals (e.g., Sig.) from the detector 718, which may help guide how it adjusts the virtual environment. The stimulation engine 724 may determine if a stimulation should be provided (and if so what type) based on if the signal trigger is active or not. For example, if the signal trigger is a logical high, then a stimulation may be provided.

The decoder 700 may also include a back end interface which allows adjustment of the operation of the decoder 700. The interface represents various settings which may be changed or adjusted in some embodiments of the XR-NPF system. For example, if the XR-NPF system is being used for clinical and/or research applications, the interface may represent a view presented to the clinician and/or researcher that allows them to adjust the operation of the decoder 700. For example, the interface may allow updating of some parameters during runtime, including channel selection, bandpass filter settings, bandpower and threshold calculation settings. The interface may include visual representations of the operation of the decoder 700, which may be useful to monitor the operation of the decoder 700. In some embodiments, the interface may be presented on a display separate from the one or more displays in the XR system, such as on a general purpose computer monitor facing a clinician/researcher.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. A method comprising:
presenting a virtual environment to a user with an extended reality (XR) system;
determining an interaction of the user's with the virtual environment;
generating a stimulation pattern responsive to the user's interaction with the virtual environment; and
applying stimulation to the user's nervous system with a stimulation system based on the stimulation pattern.

2. The method of claim 1, further comprising applying a perceptual stimulation to the user's nervous system with the stimulation system.

3. The method of claim 2, further comprising applying the stimulation to a sensory region of the user's brain.

4. The method of claim 1, further comprising applying the stimulation through implanted electrodes.

5. The method of claim 1, further comprising selecting the stimulation pattern based on a type of the interaction, a type of virtual object being interacted with, or combinations thereof.

6. The method of claim 1, further comprising generating the stimulation pattern responsive to the user grasping a virtual object in the virtual environment.

7. The method of claim 6, further comprising generating a first stimulation pattern responsive to grasping a first type of virtual object and generating a second stimulation pattern responsive to grasping a second type of object.

8. The method of claim 7, further comprising not generating the stimulation pattern or applying the stimulation responsive to grasping a third type of virtual object.

9. The method of claim 1, further comprising training the user's nervous system to associate the stimulation with the interaction performed by the user in the virtual environment.

10. The method of claim 1, wherein applying the stimulation includes modulating an amplitude of a sequence of stimulation pulses over time based on the stimulation pattern.

11. The method of claim 1, further comprising:
receiving information from sensors which measure activity in the user's nervous system;
decoding the information; and
determining the interaction with the virtual environment based on the decoded information.

12. A system comprising:
an extended reality (XR) system configured to present a virtual environment to a user;
a neural interface configured to deliver stimulation to a nervous system of the user based on a stimulation pattern; and
a computing system configured to generate the stimulation pattern responsive to an interaction of the user with the virtual environment and provide the stimulation pattern to the neural interface, wherein the neural interface is configured to apply the stimulation pattern through the at least one electrode to the nervous system of the user.

13. The system of claim 12, wherein the XR system includes at least one controller configured to allow the user to interact with the virtual environment.

14. The system of claim 12, further comprising at least one sensor configured to measure activity in the nervous system of the user,
wherein the computing system includes a decoder configured to determine an interaction with the virtual environment based on decoding the measured activity from the sensor.

15. The system of claim 12, wherein the XR system includes a headset configured to be worn by the user.

16. The system of claim 12, wherein the at least one electrode is implanted in the user.

17. A system including:
an extended reality (XR) headset;
a neural interface including at least one stimulation electrode;
at least one processor; and
computer readable non-transitory media loaded with instructions, which when executed by the at least one processor cause the system to:
generate a virtual environment and provide information related to the virtual environment to the XR system;
receive input information from the XR system;
determine an interaction with the virtual environment based on the input information; and
provide a stimulation pattern to the neural interface responsive to the interaction, wherein the neural interface is configured to apply voltage to the at least one stimulation electrode based on the stimulation pattern.

18. The system of claim 17, further comprising at least one controller configured to generate at least part of the input information.

19. The system of claim 17, wherein the input information includes motion tracking information.

20. The system of claim 17, further comprising at least one sensor configured to measure activity in a user's nervous system, wherein the instructions, when executed by the at least one processor further cause the system to decode the measured activity, wherein the decoded activity is at least part of the input information.

21. The system of claim 17, wherein the at least one stimulation electrode is implanted in a user.

22. The system of claim 21, wherein the at least one stimulation electrode is implanted in a sensory region of the user's brain.

23. The system of claim 17, wherein the instructions when executed by the at least one processor, further cause the system to select the stimulation pattern from a plurality of stimulation patterns, wherein the selection is based on a type of the interaction, a type of virtual object the interaction is with, or combinations thereof.
